(19) United States
(12) Patent Application Publication
(10) Pub. No.: US 2023/0096683 A1
(43) Pub. Date: Mar. 30, 2023

(54) PATELLAR IMPLANT

(71) Applicant: EPISURF IP-MANAGEMENT AB, Stockholm (SE)

(72) Inventors: Johan JULIN, Stockholm (SE); Ingrid BRATT, Solna (SE); Katarina FLODSTRÖM, Danderyd (SE); Leif RYD, Malmö (SE)

(73) Assignee: EPISURF IP-MANAGEMENT AB, Stockholm (SE)

(21) Appl. No.: 17/486,538

(22) Filed: Sep. 27, 2021

(51) Int. Cl. A61F 2/38; A61F 2/46; A61F 2/30

(52) U.S. Cl. CPC A61F 2/3877; A61F 2/3859; A61F 2/461; A61F 2/30942; A61F 2002/30878; A61F 2002/30617; A61F 2310/00023; A61F 2310/00029; A61F 2002/4632

(57) ABSTRACT

In accordance with one or more embodiments herein, a patellofemoral implant arrangement 200 for repairing damage in a patellofemoral articulation of a patient is provided. The patellofemoral implant arrangement 200 comprises a femoral trochlear implant 250, comprising an articulating surface 255, and a patellar implant 300, configured to be inserted, preferably with press-fit, into a recess 620 in a patella 600 in such a way that the perimeter of an articulating surface 310 of the patellar implant 300 does not extend beyond a surrounding articulating surface of the patella 600. The articulating surfaces 255, 310 of the femoral trochlear implant 250 and the patellar implant 300 are designed to allow that they at least partly interact with each other when the implants 250, 300 are implanted into the knee joint and the patella 600 lies in the intercondylar groove of the femur. Preferably, the articulating surface 255 of the femoral trochlear implant 250 is a metal or ceramic surface; and the articulating surface 310 of the patellar implant 300 is not a metal or ceramic surface. The articulating surface 310 of the patellar implant 300 may be designed to correspond to the curvature of a simulated healthy articulating surface of the undamaged patella 600 at the site of diseased cartilage. The contour curvature of the articulating surface 310 may be generated based on the determined surface curvature of the cartilage and/or the subchondral bone in a predetermined area comprising and surrounding the site of diseased cartilage and/or bone in the patella 600, to mimic the original, undamaged, articulating surface of the patella 600.

TECHNICAL FIELD

The present disclosure relates generally to in-lay implants suitable for repairing damage in the patella of a patient, especially damage in the articular surface of the patella.

### BACKGROUND

Pain and overuse disorders of the joints of the body is a common problem, and one of the most important joints which is liable to wear and disease is the knee. The knee provides support and mobility, and is the largest and strongest joint in the body. Pain in the knee can be caused by for example injury, arthritis or infection.

The knee joint consists of four bones (femur, tibia, patella and fibula) with three articulations: the tibiofemoral articulation (between the convex femoral condyles and the concave tibial condyles), the patellofemoral articulation (where the patella lies in the intercondylar groove of the femoral trochlea), and the tibiofibular articulation (between the tibia and the fibula). The tibia, femur and patella are covered in articular cartilage, and the normal function of the knee joint depends upon this. The friction between the cartilage and the surrounding parts of the joint is very low, which facilitates movement of the joints under high pressure. The cartilage is however prone to damage due to disease, injury or chronic wear. Moreover, the cartilage does not readily heal after damages, as opposed to other connective tissue, and if healed, durable hyaline cartilage is often replaced by less durable fibrocartilage. This means that damages of the cartilage have a tendency to gradually become worse. It is therefore important to have efficient means and methods for repairing damaged cartilage in knee joints.

The advantages of using implants for repairing damaged cartilage have stimulated the development of small joint implants, suitable for repair of small injuries to cartilage and/or underlying bone, that have a minimal influence on the surrounding parts of the joint. Such small implants are often designed with an implant body that may be formed as a plate with a wear resistant articulating surface for facing the articulate side of the joint, and a bone contacting surface for facing the bone below the damaged part of cartilage. The shape and the curvature of the articulating surface of the implant may be designed to be similar to the shape and the curvature of the part of the joint where the implant is inserted. Such small implants are often designed with a mushroom-like shape, having an implant body, or head, and a peg, or rod, projecting from the bone contacting side of the implant body for fastening the implant to the bone.

Patellar cartilage lesions are common, and often occur in conjunction with lesions on the femoral trochlea. Since the patella is such a small bone, patellar implants are normally in the form of patellar resurfacing implants, where the whole patellar surface is resected, before the patellar implant is attached to the resected surface. U.S. Pat. Nos. 6,602,292, 10,893,948 and 10,874,408 describe such patellar resurfacing implants.

WO2020092335 proposes the use of a dome-shaped patellar implant comprising dimples.

### PROBLEMS WITH THE PRIOR ART

The use of patellar resurfacing implants normally requires the resection of the whole patellar surface, and this is a rather complicated procedure.

Therefore, there is a need for improved implants suitable for repairing damage in the patella of a patient.

### SUMMARY

The above described problem is addressed by the claimed patellar implant for repairing damage in a patellofemoral articulation of a patient. The patellar implant is preferably adapted to be inserted, preferably with press-fit, into a recess in a patella in such a way that the perimeter of an articulating surface of the patellar implant does not extend beyond a surrounding articulating surface of the patella. The articulating surface of the patellar implant is designed to correspond to the curvature of a simulated healthy articulating surface of the undamaged patella at a site of diseased cartilage and/or bone. The contour curvature of the articulating surface is generated based on a determined surface curvature of the cartilage and/or the subchondral bone in a predetermined area at the site of diseased cartilage and/or bone, to mimic the original, undamaged, articulating surface of the patella. This enables the repairing of damage to the patella without the need for a resection of the whole patellar surface.

In embodiments, the articulating surface of the patellar implant comprises a positioning mark. This makes it easier to accomplish a correct rotational positioning of the patellar implant during surgery, which is important because the articulating surface of the patellar implant will in most situations not be rotationally symmetric. The positioning mark may in embodiments extend also to the side of the patellar implant.

In embodiments, the patellar implant comprises a bone contacting surface comprising an implant peg extending from a bone contacting surface, and one or more implant cavities with openings towards the bone contacting surface. This enables a secure fastening of the patellar implant to the patella, especially if the recess in the patella is filled with an adhesive, such as e.g. bone cement, that during insertion of the implant is pressed into the one or more implant cavities.

In embodiments, the patellar implant comprises one or more side recesses, and adhesive guides arranged to guide adhesive to the side recesses when the patellar implant is inserted into the recess in the patella.

The above described problem is also addressed by the claimed patellofemoral implant arrangement for repairing damage in a patellofemoral articulation of a patient, the claimed patellofemoral implant arrangement comprising a femoral trochlear implant and a patellar implant. The patellar implant is preferably configured to be inserted, preferably with press-fit, into a recess in a patella in such a way that the perimeter of an articulating surface of the patellar implant does not extend beyond a surrounding articulating surface of the patella. The articulating surfaces of the femoral trochlear implant and the patellar implant are preferably designed to allow that they at least partly interact with each other when the implants are implanted into the knee joint and the patella lies in the intercondylar groove of the femur. Preferably, the articulating surface of the femoral trochlear implant is a metal or ceramic surface, and the articulating surface of the patellar implant is not a metal or ceramic surface. This enables the repairing of damage in a patellofemoral articulation of a patient with an implant arrangement that avoids a metal-on-metal interface.

In embodiments, the articulating surface of the femoral trochlear implant comprises titanium, titanium nitride, and/or a cobalt-chromium alloy. Such materials are very suitable for a femoral trochlear implant.

In embodiments, the articulating surface of the patellar implant comprises a polymer material, such as polyethylene, e.g. the polyethylene UHMWPE (e.g. cross-linked UHMWPE or vitamin E enhanced UHMWPE). This avoids a very hard surface, such as a metal or ceramic surface, interfacing with another very hard surface, creating e.g. a metal-on-metal interface between the implants. The main body of the patellar implant may be manufactured from metal or ceramic, but the articulating surface preferably comprises a polymer material, such as polyethylene, e.g. the polyethylene UHMWPE. If the bone contacting surface of the patellar implant is a non-porous metal or ceramic surface, it may be advantageous to coat the bone contacting surface with an osseointegrating and/or bioactive material, such as e.g. hydroxyapatite.

The patellar implant may be the above described patellar implant, but it may also be a standardized patellar implant, selected from a predefined set of standardized patellar implants having varying dimensions.

The above described problem is further addressed by the claimed patellar surgical kit. The patellar surgical kit preferably comprises the above described patellar implant, a patellar guide tool, and an insert tool, which may e.g. be a mandrel and/or a patellar clamp. The patellar guide tool may comprise a cartilage contact surface configured to have a shape and contour that is designed to correspond to and to fit the contour of the cartilage or the subchondral bone of the patella in a predetermined area at a site of diseased cartilage and/or bone. The insert tool may be configured to be used for inserting the patellar implant into a suitably shaped recess that has been e.g. drilled or milled into the patella, and it may have an implant engaging portion that has a surface curvature that substantially corresponds to the surface curvature of the articulating surface of the patellar implant.

The above described problem is further addressed by the claimed patellofemoral surgical kit. The patellofemoral surgical kit preferably comprises the above described patellofemoral implant arrangement, a patellar guide tool for the patellar implant, a trochlear guide tool for the femoral trochlear implant, and an insert tool, which may e.g. be a mandrel and/or a patellar clamp. The patellar guide tool for the patellar implant may comprise a cartilage contact surface configured to have a shape and contour that is designed to correspond to and to fit the contour of the cartilage or the subchondral bone of the patella in a predetermined area at a site of diseased cartilage and/or bone. The trochlear guide tool for the femoral trochlear implant may comprise a cartilage contact surface configured to have a shape and contour that is designed to correspond to and to fit the contour of the cartilage or the subchondral bone of the femur in a predetermined area at a site of diseased cartilage and/or bone. The insert tool may be configured to be used for inserting the patellar implant into a suitably shaped recess that has been e.g. drilled or milled into the patella, and it may have an implant engaging portion that has a surface curvature that substantially corresponds to the surface curvature of the articulating surface of the patellar implant. Preferably, the patellar guide tool for the patellar implant and/or the trochlear guide tool for the femoral trochlear implant comprise visual markings, so that they are visually distinct from each other. In this way, it will be clear to the surgeon which guide tool to use for which implant.

The above described problem is also addressed by the claimed system for customizing a patellar implant for repairing damage in a patellofemoral articulation of a patient. The patellar implant is preferably configured to be inserted, preferably with press-fit, into a recess in a patella in such a way that the perimeter of an articulating surface of the patellar implant does not extend beyond a surrounding articulating surface of the patella. The system preferably comprises at least one processor configured to: obtain a three-dimensional image representation of the patella based on medical images generated using a medical imaging system; determine damage to the patella by analyzing medical images generated using a medical imaging system; and determine the shape and dimensions of a customized patellar implant suitable for repairing said determined damage, using said three-dimensional image representation of the patella, wherein the contour curvature of the articulating surface is generated based on the determined surface curvature of the cartilage and/or the subchondral bone in a predetermined area at a site of diseased cartilage and/or bone, to mimic the original, undamaged, articulating surface of the patella. This enables the repairing of damage to the patella without the need for a resection of the whole patellar surface.

In embodiments, the at least one processor is configured to determine the shape and dimensions of the customized patellar implant by simulating a healthy articulating patellar surface at the site of the determined damage, including designing the surface of the customized patellar implant to match said simulated healthy articulating patellar surface.

In embodiments, the healthy articulating patellar surface is simulated based on the determined surface curvature of the cartilage and/or the subchondral bone in a predetermined area comprising and surrounding the determined damage.

The above described problem is further addressed by the claimed method for customizing a patellar implant for repairing damage in a patellofemoral articulation of a patient. The patellar implant is preferably configured to be inserted, preferably with press-fit, into a recess in a patella in such a way that the perimeter of an articulating surface of the patellar implant does not extend beyond a surrounding articulating surface of the patella. The method preferably comprises: obtaining a three-dimensional image representation of the patella based on medical images generated using a medical imaging system; determining damage to the patella by analyzing medical images generated using a medical imaging system; and determining the shape and dimensions of a customized patellar implant suitable for repairing said determined damage, using said three-dimensional image representation of the patella.

In embodiments, the shape and dimensions of the customized patellar implant are determined by simulating a healthy articulating patellar surface at the site of the determined damage, including designing the surface of the customized patellar implant to match said simulated healthy articulating patellar surface.

In embodiments, the healthy articulating patellar surface is simulated based on the determined surface curvature of the cartilage and/or the subchondral bone in a predetermined area comprising and surrounding the determined damage.

The above described problem is also addressed by a non-transitory machine-readable medium on which is stored machine-readable code which, when executed by a processor, controls the processor to perform any one of the above described methods.

The above described problem is further addressed by the claimed method for inserting a patellar implant comprising a positioning mark at a site of determined damage in a patella. The method preferably comprises: attaching a guide tool to the articulating surface of the patella, the guide tool comprising a cartilage contact surface configured to have a shape and contour that is designed to correspond to and to fit the contour of the cartilage or the subchondral bone of the patella in a predetermined area at a site of diseased cartilage and/or bone; creating a recess in the patella, e.g. by drilling or milling the recess, using the guide tool; making a marking on the cartilage at the side of the recess in the patella; removing the guide tool from the patella; applying an adhesive in the recess in the patella; rotating the patellar implant so that the positioning mark is aligned with the marking on the cartilage; inserting the patellar implant into the recess; pressing the patellar implant into the recess in the patella, using an insert tool; and removing the insert tool.

In embodiments, the method is adapted to be automatically performed by a robot.

The definition that the perimeter of the implant “does not extend beyond” a surrounding articulating surface covers if the perimeter of the implant surface after insertion into the recess is approximately level with the surrounding articulating surface. In practice, it is however often desired that the perimeter of the implant surface is slightly (e.g. 0.5-1 mm) lower than the surrounding articulating surface. This ensures that there will be no sharp implant edges sticking out of the surrounding cartilage, even if the surrounding cartilage is compressed when loaded; such sharp edges may be painful for the patient. This also enables a thin layer of tissue to grow on the surface of the implant.

The definition that the articulating surfaces of the femoral trochlear implant and the patellar implant are designed to allow that they “at least partly interact with” each other also covers if the articulating surfaces are designed to allow interaction only during certain types of movements of the knee joint. They do not necessarily have be designed to allow interaction during all types of movements of the knee joints, but they must be designed to allow interaction during some types of movements of the knee joint.

The medical imaging system may e.g. be a magnetic resonance imaging (MRI) system, an x-ray imaging system, an ultrasonic imaging system, a fluoroscopic imaging system and/or a computer tomography (CT), e.g. a CBCT, system. The medical images may be a number of images in a series captured during a process of scanning through different layers of the anatomical joint or part of it using a medical imaging system.

The processor may in some embodiments comprise several different processors which together perform the claimed functions.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 shows a schematic view of a system for customizing a patellar implant, in accordance with one or more embodiments described herein.

FIGS. 2a-b illustrate a patellofemoral implant arrangement for repairing damage in the patellofemoral articulation, in accordance with one or more embodiments described herein.

FIGS. 3a-d illustrate a patellar implant, in accordance with one or more embodiments described herein.

FIGS. 4a-c illustrate a patellar clamp, in accordance with one or more embodiments described herein.

FIGS. 5a-b illustrate a patellar guide tool, FIGS. 5c-d illustrate the drilling of a recess for a patellar implant, and FIG. 5e illustrates a surgical kit, in accordance with one or more embodiments described herein.

FIGS. 6a-b illustrate a trochlear guide tool, and FIG. 6c illustrates a guide tool for a twin implant, in accordance with one or more embodiments described herein.

FIG. 7 shows the patellofemoral implant arrangement in accordance with one or more embodiments described herein in a model of the knee joint.

FIG. 8 is a schematic flow diagram for a method for customizing a patellar implant, in accordance with one or more embodiments described herein.

FIG. 9 is a schematic flow diagram for a method for inserting a patellar implant in a patella, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

### Introduction

The primary function of the patella is to work as a mechanical pulley increasing the efficacy of the quadriceps muscle. The patella is a part of a gliding joint and performs movements in several directions. During a normal knee flexion, the patella first glides medially to center itself in the trochlear groove while tilting 5-7 degrees laterally. Further in the flexion, the patella tracks over to the lateral side. A normal patella glides about 3 mm medially and laterally from its center point during motion.

The stress on the articulating tissue is dependent on the contact area between patella and femur. Small contact area and high applied forces result in high patellofemoral stresses which can be harmful for the cartilage. During knee flexion, different articulating patella surface areas are in contact with the femoral cartilage.

The present disclosure relates generally to in-lay implants suitable for repairing damage in the patellofemoral articulation, where the patella lies in the intercondylar groove of the femur. Embodiments of the disclosed solution are presented in more detail in connection with the figures.

System architecture

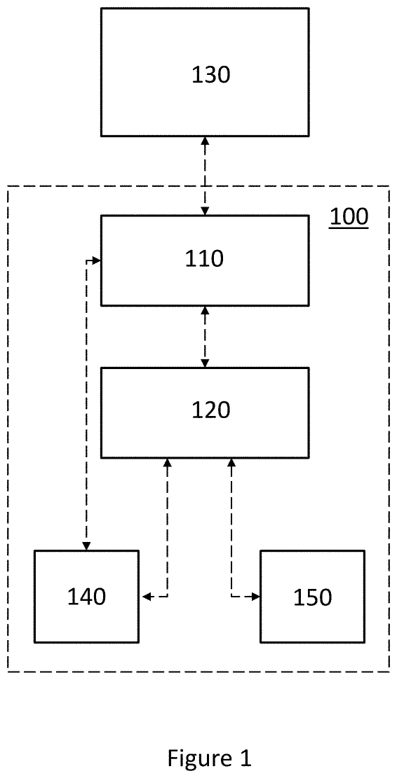

FIG. 1 shows a schematic view of a system 100 for customizing a patellar implant for repairing damage in a patellofemoral articulation of a patient. The patellar implant is preferably configured to be inserted, preferably with press-fit, into a recess in a patella in such a way that the perimeter of an articulating surface of the patellar implant does not extend beyond the surrounding articulating surface of the patella. According to embodiments, the system 100 comprises a display 140, at least one manipulation tool 150, and a storage media 110, configured to receive and store image data and parameters. In some embodiments, the system 100 is communicatively coupled to a medical imaging system 130. The medical imaging system 130 may be configured to capture or generate medical images, e.g. radiology images such as X-ray images, ultrasound images, computed tomography (CT), e.g. CBCT, images, nuclear medicine including positron emission tomography (PET) images, and magnetic resonance imaging (MRI) images. The storage media 110 may be configured to receive and store medical images from the medical imaging system 130. In embodiments, medical images are uploaded into the storage media 110 by personnel at a medical care facility, preferably the medical care facility where the medical imaging takes place. Medical images may however also be uploaded into the storage media 110 by another medical care facility, or by other authorized personnel. The uploading of the medical images may also be an automatic uploading directly from one system to another.

In one or more embodiments, the system 100 comprises at least one processor 120 configured to: obtain a three-dimensional image representation of the patella based on medical images generated using a medical imaging system 130; determine damage to the patella by analyzing medical images generated using a medical imaging system 130; and determine the shape and dimensions of a customized patellar implant suitable for repairing said determined damage, using the three-dimensional image representation of the patella. The contour curvature of the articulating surface of the patellar implant may e.g. be generated based on the determined surface curvature of the cartilage and/or the subchondral bone in a predetermined area at a site of diseased cartilage and/or bone, to mimic the original, undamaged, articulating surface of the patella.

In one or more embodiments, the at least one processor 120 is configured to determine the shape and dimensions of the customized patellar implant 300 by simulating a healthy surface at the site of the determined damage, including designing the surface of the customized patellar implant 300 to match the simulated healthy surface. The healthy surface may e.g. be simulated based on the determined surface curvature of the cartilage and/or the subchondral bone in a predetermined area comprising and surrounding the determined damage. The determination of the shape and dimensions of a customized patellar implant 300 preferably involves designing an implant surface that corresponds to a 3D image of a simulated healthy cartilage surface.

It is desirable to simulate a healthy cartilage surface as closely as possible. If just the 3D curvature of the subchondral bone subjacent to the area of damaged cartilage is used for designing the surface of the implant 300, this does not necessarily correspond to a simulated healthy cartilage surface and mimic the original, undamaged, articulating surface of the patella 600, since the cartilage does not necessarily have uniform thickness. However, for implants in anatomical parts where the cartilage does have a substantially uniform thickness, the 3D curvature of the subchondral bone subjacent to the area of damaged cartilage may be used for designing the surface of the implant.

According to embodiments, the healthy surface is instead simulated based on the curvature of the cartilage surrounding the area of damaged cartilage. Preferably, a suitable area comprising and extending around the damaged cartilage is selected, and the curvature of the whole area is simulated in such a way that the curvature of the area which is not damaged matches the actual curvature, and a simulated healthy surface of the area of damaged cartilage is generated. The simulation may comprise an interpolation, e.g. using the Solid Works Surface Wizard or another suitable tool. The determined damage may be marked as more or less severe. Damage that is very mild may not need repairing, but severe lesions should preferably have a surface coverage of at least 90%.

In embodiments, the at least one processor 120 is configured to also output the shape and dimensions of the customized patellar implant 300 as parameters for manufacturing said customized patellar implant 300.

The at least one processor 120 may for example be a general data processor, or other circuit or integrated circuit capable of executing instructions to perform various processing operations. The at least one processor 120 may in some embodiments comprise several different processors 120 which together perform the claimed functions. In the same way, the storage media 110 may in some embodiments comprise several different storage media 110 which together perform the claimed functions.

The display 140 may be configured to receive image data for display via the processor 120, and/or to retrieve image data for display directly from the storage media 110, possibly in response to a control signal received from the processor 120 or the at least one manipulation tool 150.

The processor 120 may further be configured to perform any or all of the method steps of any or all of the embodiments presented herein.

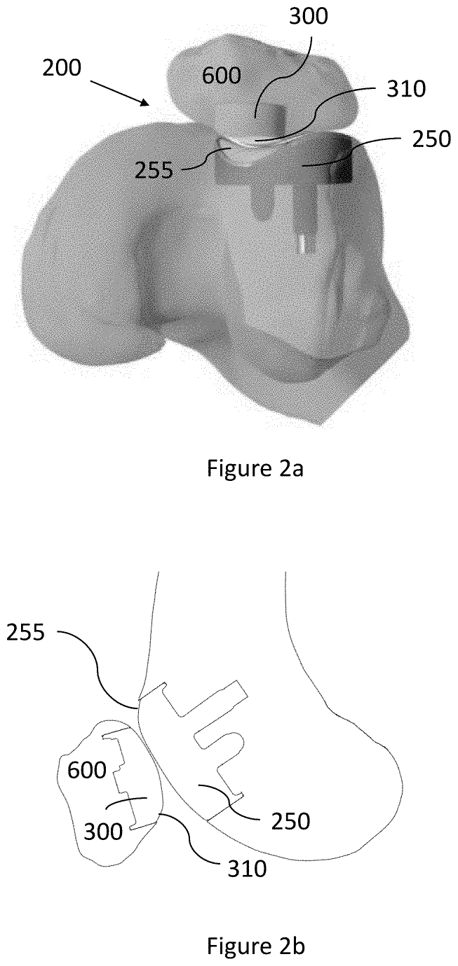

FIGS. 2a-b illustrate a patellofemoral implant arrangement 200 for repairing damage in a patellofemoral articulation of a patient. The patellofemoral implant arrangement comprises a femoral trochlear implant 250 and a patellar implant 300. The implants 250, 300 are illustrated as implanted into a knee joint, where the patella 600 lies in the intercondylar groove of the femur. The implants 250, 300 comprise articulating surfaces 255, 310 that are designed to allow that they at least partly interact with each other when the implants 250, 300 are implanted into the knee joint. The patellar implant 300 is preferably configured to be inserted, preferably with press-fit, into a recess in a patella 600 in such a way that the perimeter of the articulating surface 310 of the patellar implant 300 does not extend beyond a surrounding articulating surface of the patella 600. The definition that the patellar implant 300 “does not extend beyond” a surrounding articulating surface of the patella 600 also covers if the perimeter of the articulating surface 310 of the patellar implant 300 after insertion is approximately level with the surrounding articulating surface. In practice, it is however often desired that the perimeter of the articulating surface 310 of the patellar implant 300 is slightly (e.g. 0.5-1 mm) lower than the surrounding articulating surface. This ensures that there will be no sharp implant edges sticking out of the surrounding cartilage, even if the surrounding cartilage is compressed when loaded; such sharp edges may be painful for the patient. This also enables a thin layer of tissue to grow on the articulating surface 310 of the patellar implant 300.

The articulating surface 255 of the femoral trochlear implant 250 is preferably a metal or ceramic surface, e.g. comprising titanium (Ti), titanium nitride (TiN), and/or a cobalt-chromium (CoCr) alloy. In order to avoid a very hard surface, such as a metal or ceramic surface, interfacing with another very hard surface, creating e.g. a metal-on-metal interface, the patellar implant 300 preferably has an articulating surface 310 that is not a metal or ceramic surface. The articulating surface 310 of the patellar implant 300 is preferably a polymer surface, e.g. a surface of polyethylene, e.g. the polyethylene UHMWPE (e.g. cross-linked UHMWPE or vitamin E enhanced UHMWPE). Preferably, the whole patellar implant 300 is manufactured from the same polymer material, since this simplifies the manufacturing process.

The main body of the patellar implant 300 may be manufactured from metal or ceramic, but the articulating surface 310 preferably comprises a polymer material, such as polyethylene, e.g. the polyethylene UHMWPE. If the bone contacting surface of the patellar implant 300 is a non-porous metal or ceramic surface, it may be advantageous to coat the bone contacting surface with an osseointegrating and/or bioactive material, such as e.g. hydroxyapatite. This reduces the need for using an adhesive for to securing the patellar implant 300 to the patella 600.

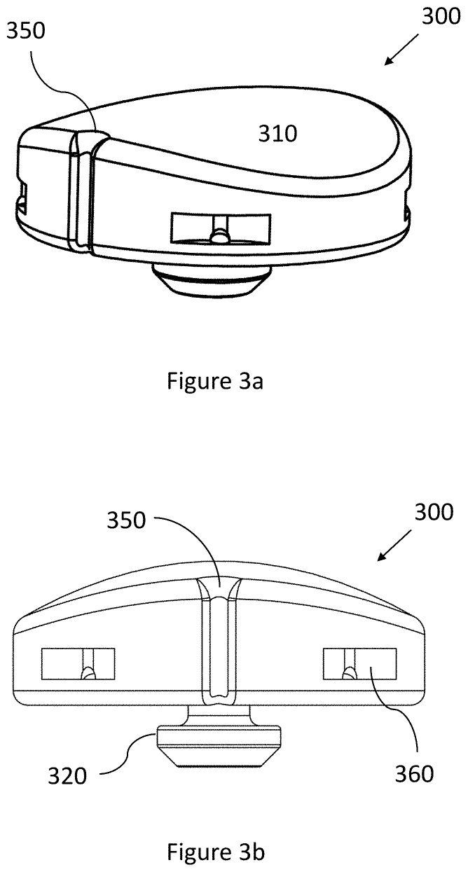

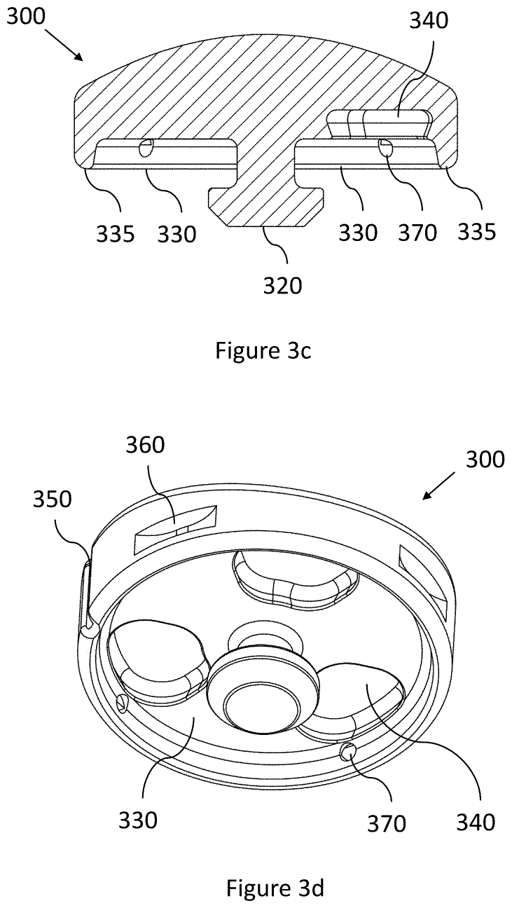

FIGS. 3a-d illustrate an embodiment of a patellar in-lay implant 300 comprising an articulating surface 310. The illustrated patellar implant 300 has an approximately cylindrical shape, but the patellar implant 300 may also have a shape that corresponds to a plurality of partly overlapping cylinders. The patellar implant 300 may comprise an implant peg 320, extending from a suitably shaped bone contacting surface 330, which in embodiments is surrounded by an implant flange 335. The patellar implant 300 preferably comprises one or more implant cavities 340 with openings towards the bone contacting surface 330. Before the patellar implant 300 is inserted into a suitably shaped recess 620 in the patella 600, the recess 620 is preferably filled with an adhesive such as e.g. bone cement. Bone cement consists of powder, most commonly polymethylmethacrylate (PMMA), mixed with liquid. When the patellar implant 300 is inserted into the recess 620, the adhesive is pressed into the one or more implant cavities 340, and the adhesive in these implant cavities 340 helps securing the patellar implant 300 to the patella 600. If an adhesive such as bone cement is used, it may not be necessary for the patellar implant 300 to be designed for press-fit in the recess 620. The use of press-fit (where the implant is slightly larger than the recess) secures the patellar implant 300 to the recess 620 regardless of whether an adhesive such as bone cement is used, but the combination of press-fit and adhesive of course secures the patellar implant 300 even more to the recess 620.

The patellar implant 300 may comprise one or more side recesses 360, which may have adhesive guides 370 that guide the adhesive to the side recesses 360 when the patellar implant 300 is inserted into the recess 620 in the patella 600. An adhesive guide 370 may e.g. be in the form of a channel, as illustrated in FIGS. 3c-d, or in the form of an indentation in the side of the patellar implant 300 up to the side recess 360, that guides the adhesive from below the patellar implant 300 up to the side recess 360. If the patellar implant 300 comprises an implant flange 335 surrounding the bone contacting surface 330, an adhesive guide 370 may be a side opening arranged in the implant flange 335. If there is no implant flange 335, an adhesive guide 370 may e.g. be an opening extending from an implant cavity 340 to a side recess 360. The adhesive in the side recesses 360 helps securing the patellar implant 300 to the patella 600.

The bone contacting surface 330 of the patellar implant 300 may be coated with a bioactive material, such as e.g. hydroxyapatite. In such a case, it may not be necessary to use an adhesive. It may however be difficult to coat a polymer material with a bioactive material, so the use of a bioactive coating on the bone contacting surface is simplified if the body of the patellar implant 300 is manufactured from metal or ceramic, even though the articulating surface 310 comprises a polymer material.

The patellar implant 300 may also comprise a positioning mark 350, preferably positioned on the articulating surface 310. The positioning mark 350 may in embodiments extend also to the side of the patellar implant 300, e.g. in the form of an indentation 350 in the side of the patellar implant 300.

The surface curvature of the articulating surface 310 of the patellar implant 300 preferably corresponds as closely as possible to the surface curvature of the undamaged patella 600. By analyzing the surface curvature of the cartilage and/or the subchondral bone in a predetermined area comprising and surrounding the site of diseased cartilage, it is possible to simulate a healthy articulating surface of the undamaged patella 600 and mimic the original, undamaged, articulating surface of the patella 600. The image data may be analyzed in a data processing system to identify and determine physical parameters for the cartilage damage. The physical parameters to be determined may comprise the presence, the location and the size and shape of the cartilage damage, as well as curvature of the surface contour of the cartilage or the subchondral bone in an area of the cartilage damage.

When such a healthy articulating patellar surface has been simulated, it is possible to design an individualized patellar in-lay implant 300 with an articulating surface 310 that corresponds to the simulated healthy articulating patellar surface.

However, it is also possible to select the best matching predefined surface from a limited number of different predefined surfaces. This enables the use of standardized patellar implants 300. In this way, a set of standardized patellar implants 300 of different dimensions may be manufactured and stored, to be later used for repairing damage in the patellofemoral articulation. Even if the femoral trochlear implant 250 is entirely individualized, and manufactured for a specific patient, it may still be advantageous to use a standardized patellar implant. This is because the manufacturing of entirely individualized patellar implants 300 is normally much more expensive than the batch-wise manufacturing of patellar implants 300, due to the patellar implants 300 preferably not being manufactured from metal.

A standardized patellar implant 300 may in this case be selected from a predefined set of standardized patellar implants 300 having varying dimensions. The predefined set of standardized patellar implants 300 is preferably created by analyzing dimensional data from stored images of the patella 600 from a large number of different patients. The standardized patellar implant 300 should be selected as a standardized patellar implant 300 having dimensions that match a determined damage, thereby making it suitable for repairing the determined damage. A 3D model of the patella, visualizing the determined damage, may be used in order to determine which standardized patellar implant 300 is the best fit for repairing the determined damage.

However, even if it is possible to use a standardized patellar implant 300, there will always be cases where it cannot be ascertained that a standardized patellar implant 300 will really fit in the recess 620 in the patella 600, and repair the damage without at any point extending beyond the surrounding articulating surface of the patella 600. This may e.g. be the case for unusual types of damage to the patella 600, or unusual shapes of the patella 600. In order to ascertain that the patellar implant 300 will really fit in the recess 620 in the patella 600, and repair the damage without at any point extending beyond the surrounding articulating surface of the patella 600, it is necessary to design an individualized patellar in-lay implant 300 with an articulating surface 310 that corresponds to the simulated healthy articulating patellar surface.

The patellar implant 300 may be used alone, or together with a femoral trochlear implant 250 in a patellofemoral implant arrangement 200, as illustrated in FIGS. 2a-b.

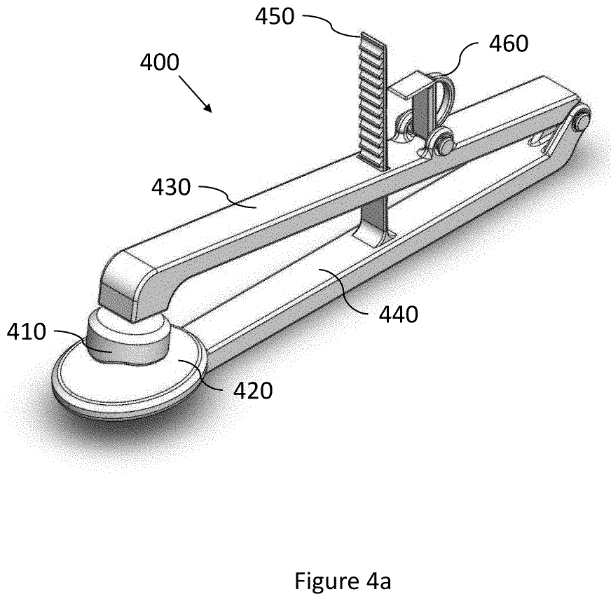

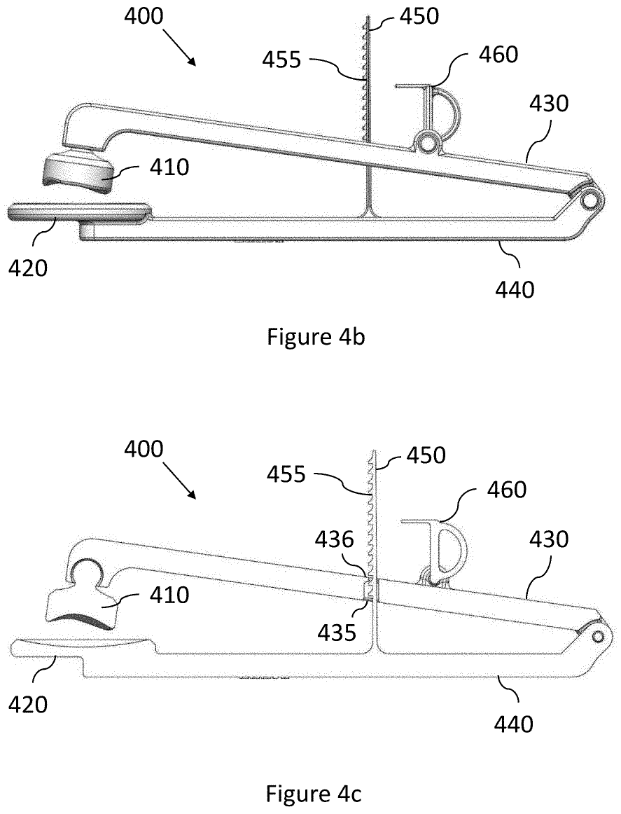

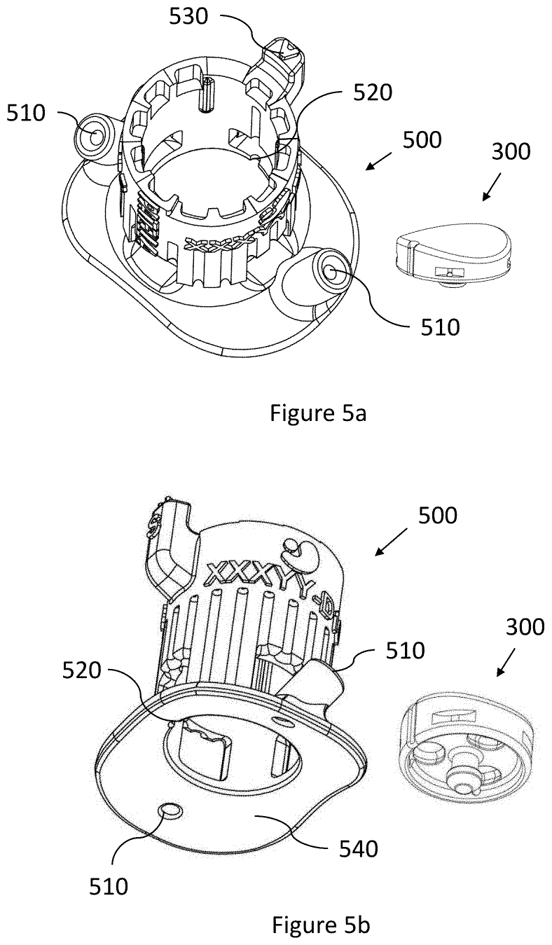

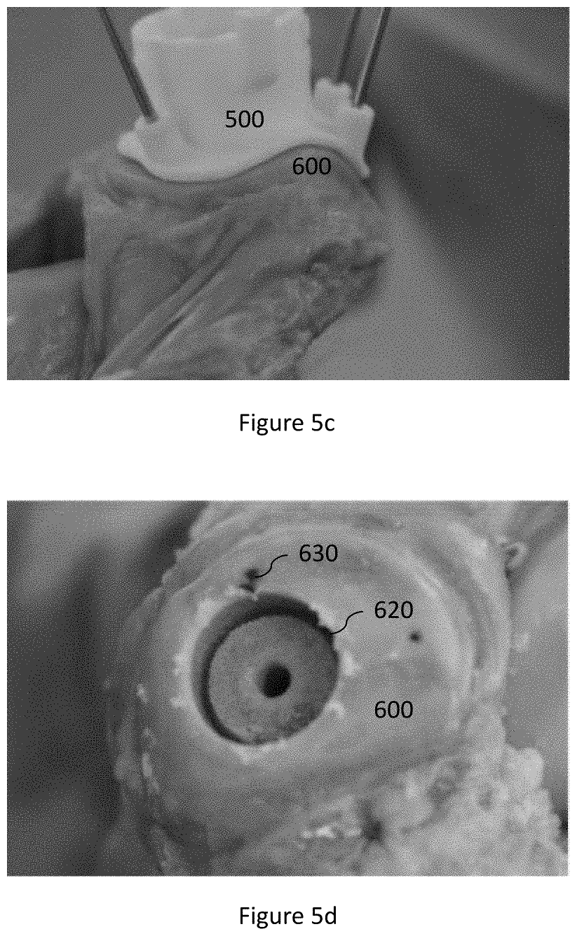

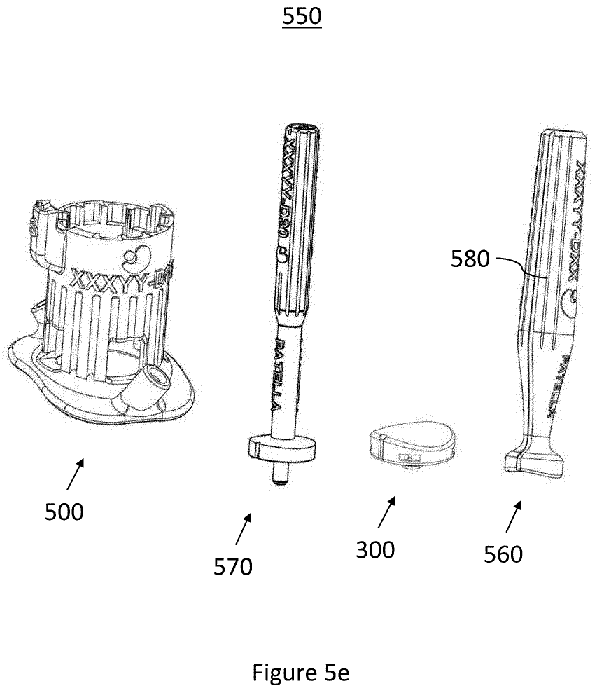

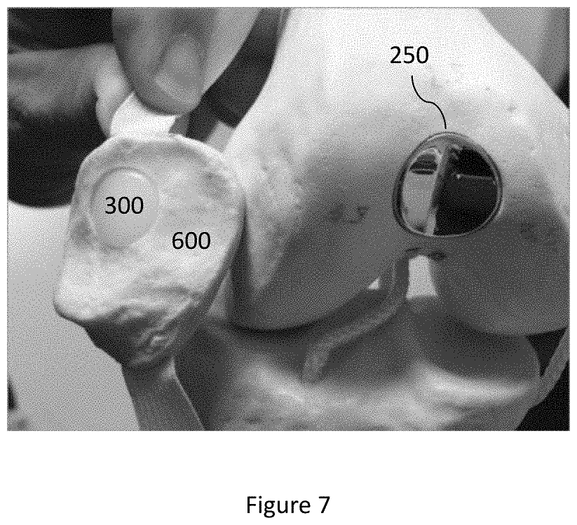

FIGS. 4a-c illustrate an insert tool in the form of a patellar clamp 400, to be used for inserting the patellar in-lay implant 300 into a suitably shaped recess 620 that has been e.g. drilled or milled into the patella 600. FIGS. 5c-d illustrate the drilling of a recess 620 for a patellar implant 300, where a guide tool 500 is used for guiding the tool for drilling or milling. The patellar guide tool 500 is illustrated in more detail in FIGS. 5a-b. FIG. 7 shows the patellofemoral implant arrangement 200, comprising the femoral trochlear implant 250 and the patellar implant 300, in a model of the knee joint. As illustrated in FIG. 7, the patella 600 is preferably inverted during the surgery, so that the articulating surface of the patella 600 becomes accessible to the surgeon.

The patellar clamp 400 preferably comprises an implant engaging portion 410, which preferably has a surface curvature that substantially corresponds to the surface curvature of the articulating surface 310 of the patellar implant 300. The patellar clamp 400 preferably also has a patella engaging portion 420, intended to be positioned on the other side of the patella 600. During surgery, the patellar clamp 400 grips around the patella 600 and presses the patellar implant 300 into the recess 620 in the patella 600. The pressing is done using levers 430, 440, which the surgeon can hold in one hand and press together as a tong. The top lever 430 is attached to the implant engaging portion 410, and the bottom lever 440 is attached to the patella engaging portion 420.

The patellar implant 300 is preferably attached to the patella 600 using an adhesive, e.g. bone cement, that is applied in the recess 620 in the patella 600 before the patellar implant 300 is inserted into the recess 620. In order to keep the patellar implant 300 pressed into the recess 620 in the patella 600 until the adhesive hardens, the patellar clamp 400 may comprise a locking mechanism 460, for keeping the patellar clamp 400 closed once the levers 430, 440 have been pressed together around the patella 600. Bone cement normally takes 10-15 minutes to harden, and it is difficult for the surgeon to press the levers 430, 440 together using exactly the same pressure for such a long time. Since the patellar implant 300 is preferably manufactured from a polymer material, it will be elastic enough for the pressure exerted by the patellar clamp 400 to be maintained also when the surgeon no longer presses the levers 430, 440 together, as long as there is some kind of locking mechanism 460 for keeping the patellar clamp 400 closed.

The locking mechanism 460 illustrated in FIGS. 4a-c interacts with an elongated part 450 comprising grooves 455. The elongated part 450 runs through a hole 435 in the top lever 430, with the grooves 455 parallel to the hole 435. When the locking mechanism 460 is engaged by being pivoted down towards the top lever 430, the top lever 430 is locked by a groove 455 being pressed against a flange 436 in the hole 435, as illustrated in FIG. 4c. Other locking mechanisms may of course also be used.

The implant engaging portion 420 is preferably articulated, in e.g. a ball joint, so that it will be able to stay level with the articulating surface 310 of the patellar implant 300 as the levers 430, 440 are pressed together.

Instead of the illustrated patellar clamp 400, other insert tools may be used to aid the insertion of the patellar implant into the recess 620 in the patella 600. It is e.g. possible to just use a mandrel 560 as an insert tool, as is commonly known for trochlear implants, especially if the patellar implant is designed with press-fit into the recess.

FIGS. 5a-b illustrate a patellar guide tool 500, which in FIG. 5c is shown attached to the articulating surface of the patella 600. The guide tool 500 preferably has a cartilage contact surface 540 that has a shape and contour that is designed to correspond to and to fit the contour of the articulating surface of the patella 600 in a predetermined area comprising and surrounding the site of diseased cartilage. Thereby, the cartilage contact surface 540 of the guide tool 500 corresponds to and fits to the surface contour of the articulating surface of the patella 600. The whole cartilage contact surface 540 does not have to correspond to the contour curvature of the articulating surface of the patella 600, it is enough if the cartilage contact surface 540 comprises at least three cartilage contact points, so that the guide tool 500 will have a stable mounting in the correct position on the patella 600. This helps ensuring that the recess 620 will be created in the exact position of the determined damage. Such cartilage contact points are preferably chosen to provide maximum support and positional stability for the guide tool 500.

The cartilage contact surface 540 of the guide tool 500 may be further stabilized by being attached to the patella 600 with one or more nails, rivets, wires or similar attachment means, as illustrated in FIG. 5c. Such additional attachment gives additional support and stability, and enables the cartilage contact surface 540 of the guide tool 500 to be as small as possible, which is especially important for the patella 600, since it is quite small. The guide tool 500 may comprise through-holes 510, through which the one or more nails, rivets, wires or similar attachment means may be inserted, in order to attach the guide tool 500 to the patella 600.

The guide tool 500 may comprise a rotational position indicator 530, which may be used to make a marking 630 on the cartilage at the side of the recess 620 in the patella 600. Such a marking 630 may then be used to correctly rotate the patellar implant 300 when the patellar implant 300 is inserted into the recess 620 in the patella 600. If the patellar implant 300 comprises a positioning mark 350, the alignment of this positioning mark 350 with the marking 630 on the cartilage at the side of the recess 620 in the patella 600 ensures that the patellar implant 300 is correctly rotated in the recess 620. The guide tool 500 is preferably configured to allow such a marking 630 to be made while the guide tool 500 is attached to the patella 600, as illustrated in FIG. 5d. The guide tool 500 may for this purpose comprise an indentation 520 at the position of the rotational position indicator 530 on the guide tool 500. The marking 630 may e.g. be added to the cartilage surface by inserting a marking pen into the indentation 520 in the guide tool 500 when the guide tool 500 is attached to the patella 600.

A correct rotational positioning of the patellar implant 300 is important because the articulating surface 310 of the patellar in-lay implant 300 will in most situations not be rotationally symmetric. An important reason for designing the articulating surface 310 of the patellar implant 300 to match the simulated healthy articulating surface of the patella 600 is to ensure that the patellar implant 300 fits smoothly in the patella 600, with no sharp edges. If the patellar implant 300 is not mounted with a correct rotational positioning, there may be sharp edges even though the articulating surface 310 of the patellar implant 300 is designed to match the simulated healthy articulating surface of the patella 600. Such sharp edges may be painful for the patient.

A marking 630 on the cartilage surface makes it easy for the surgeon to insert the patellar implant 300 with a correct rotational positioning, if the patellar implant 300 also comprises a positioning mark 350. Preferably, there is a positioning mark also on the insert tool 400, so that the implant engaging portion 410 may be correctly rotated with respect to the patellar implant 300.

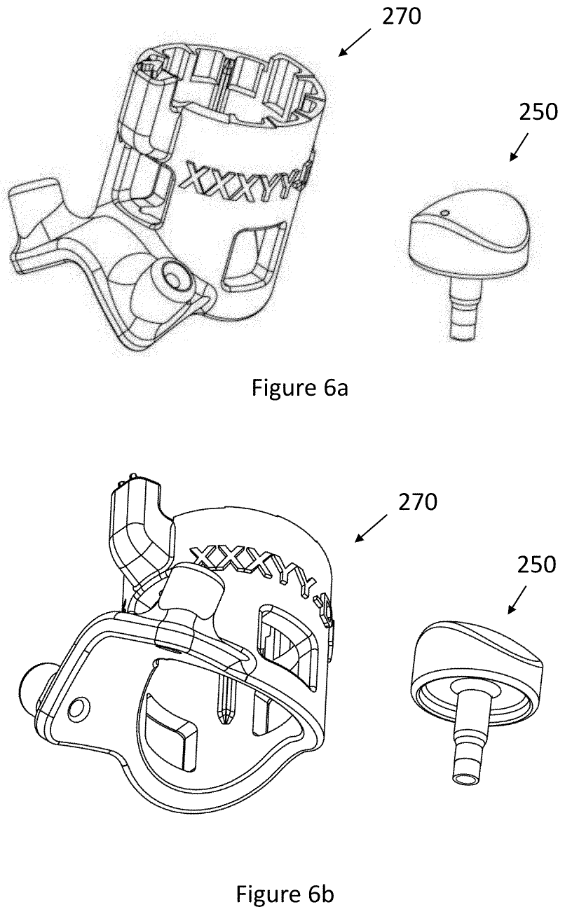

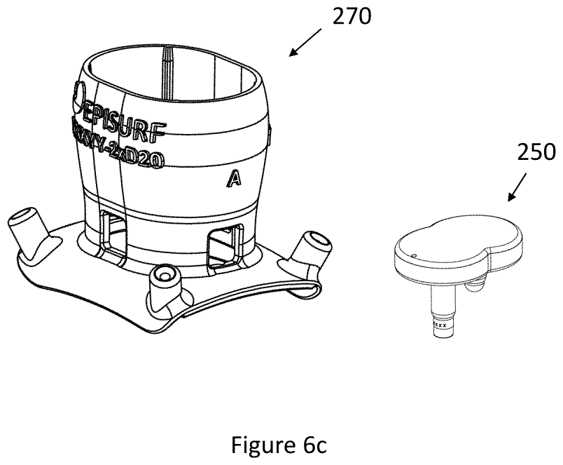

FIGS. 6a-b illustrate a trochlear guide tool 270, similar to the patellar guide tool 500 described above, and FIG. 6c illustrates a guide tool 270 for a twin implant 250 (an implant which has a shape that corresponds to two partly overlapping cylinders).

In order to repair the damaged cartilage in the patella 600, a patellar surgical kit 550 comprising the above described patellar implant 300, the above described patellar guide tool 500, and an insert tool may be used. Even if the patellar implant 300 is an implant selected from a predefined set of standardized implants having varying dimensions, it is still preferred to use a customized guide tool 500, having a cartilage contact surface 540 configured to have a shape and contour that is designed to correspond to and to fit the contour of the cartilage or the subchondral bone in a predetermined area comprising and surrounding a site of diseased cartilage, since this will ensure that the guide tool 500 will have a stable mounting in the correct position on the patella 600. This helps ensuring that the recess 620 will be created in the exact position of the determined damage. FIG. 5e illustrates a patellar surgical kit 550 comprising a patellar guide tool 500, an implant dummy 570, a patellar implant 300, and a mandrel 560 to aid insertion of the patellar implant 300 into the recess 620.

When there is damage in both the femur and the patella 600, a patellofemoral surgical kit may instead be used. The patellofemoral surgical kit may comprise the above described patellofemoral implant arrangement 200, the above described patellar guide tool 500, a trochlear guide tool 270 for the femoral trochlear implant 250, and one or more insert tools. The patellar guide tool 500 for the patellar implant and/or the trochlear guide tool 270 for the femoral trochlear implant 250 preferably comprise visual markings 580, so that they are visually distinct from each other. In this way, it will be clear to the surgeon which guide tool to use for which implant.

The insert tool may be the above described patellar clamp 400 and/or one or more mandrels 560 to aid insertion of the implant 250, 300 into the recess. A surgical kit may also comprise further instruments, such as e.g. an implant dummy 570 for verifying that the drill depth is correct before insertion of the implant, and/or one or more inserts into the patellar guide tool 500, and/or the trochlear guide tool 270 for the femoral trochlear implant 250, to enable precision in the drilling process. A drill bit, possibly adapted to the specific implant, may also be included. For a patellofemoral surgical kit, it is advantageous if either all the instruments associated with the femoral trochlear implant 250 or all the instruments associated with the patellar implant 300 comprise visual markings 580, so that it is visually clear which instruments belong together.

The visual markings 580 may be any type of markings that are visible to a surgeon, such as e.g. colour markings. However, the visual markings 580 may also be tactile, such as ribs, recesses or indentations on the parts of the instruments that are held by the surgeon during use. FIG. 5e illustrates markings 580 in the form of ribs on a patellar surgical kit.

### Method Embodiments

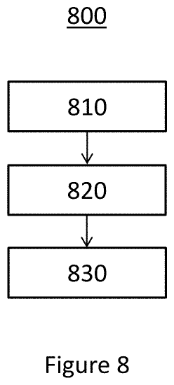

FIG. 8 is a flow diagram of embodiments of a method for customizing a patellar implant 300 for repairing damage in a patellofemoral articulation of a patient. The patellar implant 300 is configured to be inserted, preferably with press-fit, into a recess 620 in a patella 600 in such a way that the perimeter of an articulating surface 310 of the patellar implant 300 does not extend beyond a surrounding articulating surface of the patella 600. In accordance with one or more embodiments, the method 800 comprises:

Step 810: obtaining a three-dimensional image representation of the patella 600 based on medical images generated using a medical imaging system 130.

Step 820: determining damage to the patella 600 by analyzing medical images generated using a medical imaging system 130.

Step 830: determining the shape and dimensions of a customized patellar implant 300 suitable for repairing said determined damage, using said three-dimensional image representation of the patella 600.

In embodiments, the determining 830 of the shape and dimensions of the customized patellar implant 300 involves simulating a healthy articulating surface of the patella 600 at the site of the determined damage, including designing the surface of the customized patellar implant 300 to match said simulated healthy articulating patellar surface.

In embodiments, the healthy articulating patellar surface is simulated based on the determined surface curvature of the cartilage and/or the subchondral bone in a predetermined area comprising and surrounding the determined damage.

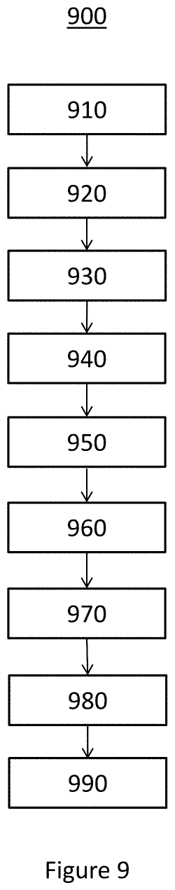

FIG. 9 is a flow diagram of embodiments of a method for inserting a patellar implant 300 comprising a positioning mark 350 at a site of determined damage in a patella, for repairing damage in a patellofemoral articulation of a patient. In accordance with one or more embodiments, the method 900 comprises:

Step 910: attaching a guide tool 500 to the articulating surface of the patella 600, the guide tool 500 comprising a cartilage contact surface 540 configured to have a shape and contour that is designed to correspond to and to fit the contour of the cartilage or the subchondral bone of the patella 600 in a predetermined area comprising and surrounding the site of diseased cartilage and/or bone in the patella 600.

Step 920: creating a recess 620 in the patella 600, e.g. by drilling or milling the recess 620, using the guide tool 500.

Step 930: making a marking 630 on the cartilage at the side of the recess 620 in the patella 600.

Step 940: removing the guide tool 500 from the patella 600.

Step 950: applying an adhesive, such as e.g. bone cement, in a recess 620 in the patella 600 and/or on the bone contacting surface 330 of the patellar implant 300.

Step 960: rotating the patellar implant 300 so that the positioning mark 350 on the patellar implant 300 is aligned with the marking 630 on the cartilage. This may take place simultaneously with step 970.

Step 970: inserting the patellar implant 300 into the recess 620.

Step 980: pressing the patellar implant 300 into the recess 620 in the patella 600, using an insert tool 400, 560.

Step 990: removing the insert tool 400.

In embodiments, the method is adapted to be automatically performed by a robot.

### Further Embodiments

Where applicable, various embodiments provided by the present disclosure can be implemented using hardware, software, or combinations of hardware and software. Also where applicable, the various hardware components and/or software components set forth herein can be combined into composite components comprising software, hardware, and/or both without departing from the claimed scope of the present disclosure. Where applicable, the various hardware components and/or software components set forth herein can be separated into sub-components comprising software, hardware, or both without departing from the claimed scope of the present disclosure. In addition, where applicable, it is contemplated that software components can be implemented as hardware components, and vice-versa. The method steps of one or more embodiments described herein may be performed automatically, by any suitable processing unit, or one or more steps may be performed manually.

Where applicable, the ordering of various steps described herein can be changed, combined into composite steps, and/or separated into sub-steps to provide features described herein.

Software in accordance with the present disclosure, such as program code and/or data, can be stored in non-transitory form on one or more machine-readable mediums. It is also contemplated that software identified herein can be implemented using one or more general purpose or specific purpose computers and/or computer systems, networked and/or otherwise.

In embodiments, there are provided a computer program product comprising computer readable code configured to, when executed in a processor, perform any or all of the method steps described herein. In some embodiments, there are provided a non-transitory computer readable memory on which is stored computer readable and computer executable code configured to, when executed in a processor, perform any or all of the method steps described herein.

In one or more embodiments, there is provided a non-transitory machine-readable medium on which is stored machine-readable code which, when executed by a processor, controls the processor to perform the method of any or all of the method embodiments presented herein.

The foregoing disclosure is not intended to limit the present invention to the precise forms or particular fields of use disclosed. It is contemplated that various alternate embodiments and/or modifications to the present invention, whether explicitly described or implied herein, are possible in light of the disclosure. Accordingly, the scope of the invention is defined only by the claims.

1. Patellar implant for repairing damage in a patellofemoral articulation of a patient, the patellar implant being adapted to be inserted, preferably with press-fit, into a recess in a patella of the patient in such a way that the perimeter of an articulating surface of the patellar implant does not extend beyond a surrounding articulating surface of the patella, wherein the articulating surface of the patellar implant is designed to correspond to the curvature of a simulated healthy articulating surface of the undamaged patella at a site of diseased cartilage and/or bone, where the contour curvature of the articulating surface is generated based on a determined surface curvature of the cartilage and/or the subchondral bone in a predetermined area at the site of diseased cartilage and/or bone, to mimic the original, undamaged, articulating surface of the patella.

2. Patellar implant according to claim 1, wherein the articulating surface of the patellar implant comprises a positioning mark.

3. Patellar implant according to claim 1, further comprising an implant peg extending from a bone contacting surface of the patellar implant, and one or more implant cavities with openings towards the bone contacting surface.

4. Patellar implant according to claim 3, further comprising one or more side recesses, and adhesive guides arranged to guide adhesive to the side recesses when the patellar implant is inserted into the recess in the patella.

5. Patellofemoral implant arrangement for repairing damage in a patellofemoral articulation of a patient, the patellofemoral implant arrangement comprising a femoral trochlear implant, comprising an articulating surface, and a patellar implant, configured to be inserted, preferably with press-fit, into a recess in a patella in such a way that the perimeter of an articulating surface of the patellar implant does not extend beyond a surrounding articulating surface of the patella, where the articulating surfaces of the femoral trochlear implant and the patellar implant are designed to allow that they at least partly interact with each other when the implants are implanted into the knee joint and the patella lies in the intercondylar groove of the femur, wherein:
the articulating surface of the femoral trochlear implant is a metal or ceramic surface; and
the articulating surface of the patellar implant is not a metal or ceramic surface.

6. Patellofemoral implant arrangement (200) according to claim 5, wherein the articulating surface of the femoral trochlear implant comprises titanium (Ti), titanium nitride (TiN), and/or a cobalt-chromium (CoCr) alloy.

7. Patellofemoral implant arrangement according to claim 5, wherein the articulating surface (310) of the patellar implant (300) comprises a polymer material.

8. Patellofemoral implant arrangement according to claim 5, wherein the patellar implant (300) comprises a bone contacting surface comprising titanium (Ti), titanium nitride (TiN), and/or a cobalt-chromium (CoCr) alloy.

9. Patellofemoral implant arrangement according to claim 5, wherein the patellar implant (300) is a patellar implant (300) according to claim 1.

10. Patellar surgical kit comprising:
a patellar implant according to claim 1;
a patellar guide tool comprising a cartilage contact surface configured to have a shape and contour that is designed to correspond to and to fit the contour of the cartilage or the subchondral bone of the patella in a predetermined area at a site of diseased cartilage and/or bone; and
an insert tool, configured to be used for inserting said patellar implant into a suitably shaped recess that has been e.g. drilled or milled into the patella.

11. Patellar surgical kit according to claim 10, wherein the insert tool has an implant engaging portion that has a surface curvature that substantially corresponds to the surface curvature of the articulating surface of the patellar implant.

12. Patellofemoral surgical kit comprising:
a patellofemoral implant arrangement according to claim 5;
a patellar guide tool for the patellar implant, comprising a cartilage contact surface configured to have a shape and contour that is designed to correspond to and to fit the contour of the cartilage or the subchondral bone of the patella in a predetermined area at a site of diseased cartilage and/or bone;
a trochlear guide tool for the femoral trochlear implant, comprising a cartilage contact surface configured to have a shape and contour that is designed to correspond to and to fit the contour of the cartilage or the subchondral bone of the femur in a predetermined area at a site of diseased cartilage and/or bone; and
one or more insert tools, configured to be used for inserting said implant into a suitably shaped recess that has been e.g. drilled or milled into the bone at a site of diseased cartilage and/or bone, wherein the guide tool for the patellar implant and/or the guide tool for the femoral trochlear implant comprise visual markings, so that they are visually distinct from each other.

13. System for customizing a patellar implant for repairing damage in a patellofemoral articulation of a patient, where the patellar implant is configured to be inserted, preferably with press-fit, into a recess in a patella in such a way that the perimeter of an articulating surface of the patellar implant does not extend beyond a surrounding articulating surface of the patella, the system comprising at least one processor configured to:
obtain a three-dimensional image representation of the patella based on medical images generated using a medical imaging system;
determine damage to the patella by analyzing medical images generated using a medical imaging system; and
determine the shape and dimensions of a customized patellar implant suitable for repairing said determined damage, using said three-dimensional image representation of the patella, wherein the contour curvature of the articulating surface is generated based on the determined surface curvature of the cartilage and/or the subchondral bone in a predetermined area at a site of diseased cartilage and/or bone, to mimic the original, undamaged, articulating surface of the patella.

14. System according to claim 13, wherein the at least one processor is configured to determine the shape and dimensions of the customized patellar implant by simulating a healthy articulating patellar surface at the site of the determined damage, including designing the surface of the customized patellar implant to match said simulated healthy articulating patellar surface.

15. System according to claim 14, wherein the healthy articulating patellar surface is simulated based on the determined surface curvature of the cartilage and/or the subchondral bone in a predetermined area comprising and surrounding the determined damage.

16. Method for customizing a patellar implant for repairing damage in a patellofemoral articulation of a patient, where the patellar implant is configured to be inserted, preferably with press-fit, into a recess in a patella in such a way that the perimeter of an articulating surface of the patellar implant does not extend beyond a surrounding articulating surface of the patella, the method comprising:
obtaining a three-dimensional image representation of the patella based on medical images generated using a medical imaging system;
determining damage to the patella by analyzing medical images generated using a medical imaging system; and
determining the shape and dimensions of a customized patellar implant suitable for repairing said determined damage, using said three-dimensional image representation of the patella.

17. Method according to claim 16, wherein the determining of the shape and dimensions of the customized patellar implant involves simulating a healthy articulating patellar surface at the site of the determined damage, including designing the surface of the customized patellar implant to match said simulated healthy articulating patellar surface.

18. Method according to claim 17, wherein the healthy articulating patellar surface is simulated based on the determined surface curvature of the cartilage and/or the subchondral bone in a predetermined area comprising and surrounding the determined damage.

19. Non-transitory machine-readable medium on which is stored machine-readable code which, when executed by at least one processor, controls the processor to perform the method of claim 16.

20. Method for inserting a patellar implant comprising a positioning mark at a site of determined damage in a patella for repairing damage in a patellofemoral articulation of a patient, the method comprising:
attaching a guide tool to the articulating surface of the patella, the guide tool comprising a cartilage contact surface configured to have a shape and contour that is designed to correspond to and to fit the contour of the cartilage or the subchondral bone of the patella in a predetermined area at the site of diseased cartilage and/or bone;
creating a recess in the patella, e.g. by drilling or milling the recess, using the guide tool;
making a marking on the cartilage at the side of the recess in the patella;
removing the guide tool from the patella;
applying an adhesive in the recess in the patella and/or on the bone contacting surface of the patellar implant;
rotating the patellar implant so that the positioning mark on the patellar implant is aligned with the marking on the cartilage;
inserting the patellar implant into the recess;
pressing the patellar implant into the recess in the patella using an insert tool; and
removing the insert tool.

21. Method according to claim 20, adapted to be automatically performed by a robot.
